(19) United States
(12) Patent Application Publication
(10) Pub. No.: US 2009/0326671 A1
(43) Pub. Date: Dec. 31, 2009

(54) MEDICAL DEVICES

(75) Inventor: Jim Schofield, Sheffield (GB)

Correspondence Address: MCKEE, VOORHEES & SEASE, P.L.C., 801 GRAND AVENUE, SUITE 3200, DES MOINES, IA, 50309-2721, US

(73) Assignee: SYMMETRY MEDICAL INC., SHEFFIELD (GB)

(21) Appl. No.: 12/445,156

(22) PCT Filed: Oct. 15, 2007
(86) PCT No.: PCT/GB07/03919

§ 371 Date: Jul. 13, 2009

(30) Foreign Application Priority Data
Oct. 13, 2006 (GB) GB 0620359.0

(51) Int. Cl. A61F 2/28; A61F 2/02; A61B 17/00; A61B 17/16; A61B 17/32; C22F 1/00

(52) U.S. Cl. 623/23.5; 623/23.76; 623/16.11; 606/1; 606/85; 606/79; 606/167; 148/512

(57) ABSTRACT

The invention to which this application relates is to the provision of medical devices such as implants for use in hips, knees or the like and also tools which may be used in surgery or for medical treatment. At least a portion of the external surface of the device is provided with a particular external surface texture which in accordance with the invention is formed by exposing said portions to at least one power beam. The exposure and relative movement between the device and the beam cause specific controlled configurations of the surface to be created without the need to add additional material and therefore allow the formation of the surfaces to the required configuration while maintaining the integrity of the device.

The invention to which this application relates is to the provision of medical devices, a term which is herein used to describe a range of articles such as joint replacement implants for use in hips, knees, shoulders, elbows, ankles, wrists, spine or the like and also tools which may be used in surgery or for medical treatment

The provision of medical devices which are formed from rapid manufacturing, powder metallurgy, metal injection moulding, precision casting, forging and machining techniques is well known. The whole device or parts of the device may be formed by these processes and the dimensions of the same are required to be closely controlled due to the tight dimensional tolerances required for their subsequent use.

It is also known that the devices or portions of the devices are required to be provided with particular external surface structural architecture. For example, for an implant, portions of the external surface of the implant may be required to have an external surface of a relatively complex porous form in order to allow and encourage bone on-growth/in-growth. Furthermore, for an implant, portions of the external surface may be required to have an external surface of a relatively complex spiked form in order to aid fixation of the implant to the bone during the surgical technique to reduce or eliminate micro-motion of the implant during service, which would otherwise hinder bone growth onto the implant. Furthermore, for surgical tools which are provided to perform cutting or rasping actions, the external surface may be required to include features which allow the rasping or cutting effect to be achieved.

In order to provide the external surface with the required structural architecture, there are conventionally many options available. One option is to perform machining operations on the external surface of the medical device. However, due to the nature of conventional machining operations the range of surface effects which can be achieved are relatively limited and production of re-entrant features are expensive. As a result of this other options are often adopted. These options generally involve the addition of a porous surface by means of coating, sintering, spraying, sputtering, etc. However a problem then exists in terms of how effectively the porous surface is joined to the actual component. Weak bonds (e.g. sintered/diffusion bonds) at the interface can lead to particles of the porous surface working loose, leading to serious problems in-vivo. When one considers an implant, the same is required to withstand prolonged use once fitted and the separation of the component from the porous surface could have catastrophic consequences. As a result of these potential problems the joining techniques used tend to be relatively complex and, as a result, expensive to achieve. Some, such as in the case of sintering, are high temperature processes which detrimentally affect the mechanically properties of the base component, notably the fatigue strength.

A further problem is that most porous coatings are high temperature processes, usually thermal spraying or sintering processes. This elevates the temperature of the base component to a point that detrimentally affects its mechanical properties. This may manifest itself in reduced wear properties, tensile strength, Young's modulus and fatigue strength, which may result in premature failure of the implant or other component. These methods also rely on weak diffusion bonds at coating/component interface. This may, in time, result in de-bonding between the coating and the implant and in turn lead to mechanical failure. A further problem is that there can be an inconsistency of form due to the random nature of the process (spraying, manual bead application) and the use of dissimilar material or different material batch to parent component, can potentially cause electrochemical corrosion and contamination potential. The random nature of such processes also limits the potential design options concerning the structural architecture.

A yet further problem is that in the manufacture of certain orthopaedic replacement hip joints it is stipulated that there should be provided a cast microstructure with no heat treatment. This ‘as-cast’ microstructure yields particular wear characteristics due to the resultant grain size and carbide morphology, thus increasing the implant longevity. Maintaining this as-cast microstructure proves problematic when such a device requires further high temperature treatments, such as diffusion bonding/sintering of porous surfaces.

The aim of the present invention is to provide a medical device and a method for forming a structural architecture on at least a portion of the same in a more efficient and effective manner than can conventionally be achieved. A further aim is to prove the manufacture of the medical device using relatively low temperature processes.

In a first aspect of the invention there is provided a medical device, wherein said device includes at least a portion of its external surface formed into a predetermined configuration by the exposure of said portion to a power beam and relative movement between the power beam and said device is controlled to form the said external surface into the predetermined configuration.

In one embodiment the power beam is a relatively high power beam and the movement between the device and the beam is relatively rapid.

In one embodiment the power beam includes a plurality of electrons directed towards the device to form the power beam. In another embodiment the power beam is a laser beam.

In one embodiment the power beam is selectively applied to said portion to provide a structural architecture of protrusions and/or networked pores and/or indents.

Typically the particular pattern of exposure and path of the relative movement is selected to achieve particular structural architecture which are required for the subsequent use of the device.

In one embodiment, the exposure to the power beam allows modification of the said portion of the surface. In one embodiment the device is initially formed and the surface portion of the same is subsequently modified by exposure to the power beam.

Typically the said surface portion which is modified is formed of the same material as the remainder of the medical device and so no additional material is required to be added to the device to form the required structural architecture. As a result no joining or bonding problems arise. Furthermore the exposure to the power beam does not adversely alter the properties of the material used to form the device and so the integrity of the device is maintained.

Typically, the exposure to the power beam fully melts the material leading to high quality welded interfaces between the surface and the remainder of the device thereby reducing the potential for any of the porous formed surface finish becoming detached from the main body.

Typically, as it is the material of the device which is, processed to form the surface finish no corrosion potential is created as no dissimilar materials are used. For this reason the likelihood of contamination from the use of secondary materials is eliminated.

In one embodiment the medical device is an implant and the surface portion is formed in a manner so as to encourage the growth and anchorage of bone to the implant and/or to improve the vascular properties of the same.

In one embodiment the medical device is a surgical tool and the surface portion is formed in a manner so as to allow an action such as cutting or rasping to be performed using the tool.

In one embodiment the portion exposed to the power beam is a portion which is subsequently to be used such as a handle of a medical device.

In one embodiment the device is an orthopaedic implant with an articulating surface, said surface exposed to a power beam prior to final machining/polishing of the device surface. In this embodiment the exposed portion material, to a given depth, is melted and rapidly re-solidified in order to remove defects and homogenise/refine the microstructure. It is found that this process allows the modified material to have enhanced tribological properties, thus increasing wear resistance and longevity of use of the device.

In one embodiment the portion is formed to have any or any combination of the following features of pores and/or re-entrant features which provide macro-interlocking of the bone and implant. In one embodiment the pores are of the range of ˜1 mm in size.

In addition or alternatively the portion can be formed with micro pores suitable for the incorporation of bone trabecula. In one embodiment this is in the range of 100-200 microns.

In addition or alternatively the pores or surface texture may be suitable for the attachment of bone building cells (osteoclasts & osteoblasts) and in one embodiment may be in the range of 10-20 microns.

In one embodiment the device beam to which the device is exposed is of the type disclosed in Patent GB2375728.

Typically the power beam is moved in a traverse direction relative to the device surface so as to expose the portion of the device to the power beam. Typically the movement of the power beam and/or device is controlled to be in a predefined manner so as to melt and displace and form the material at the surface of the portion to configure the same.

In one embodiment the power beam is continuous or alternatively may be pulsed.

Typically the duration and movement of the power beam and/or movement of the device are closely controlled in order to allow the configuration of the device surface to be repeatable.

In a further aspect of the invention there is provided a medical device which has at least a portion of its external surface modified after the device has been initially formed, said modification achieved by the selective exposure of said portion to a power beam, and controlled relative movement between the beam and said portion.

In a yet further aspect of the invention there is provided a method of forming a medical device, said method comprising the steps of forming the device from a metal or metal alloy into an initial form and wherein at least a portion of the surface is exposed to a power beam and relative movement between the device and power beam causes modification of the portion to create a surface of the desired form.

In accordance with the invention there is provided a high temperature process in confined areas in that the material melted by the process is in relatively small areas. Therefore the main body of the component remains at temperatures that do not negatively affect the mechanical properties of the device.

Typically, due to the short duration of the metal melting, rapid solidification results in a textured zone with a fine grained and homogenous microstructure, and therefore has superior mechanical properties.

Typically due to fill material melting, the resulting connection between the structure and the base component has superior mechanical properties, i.e. as strong as a weld and the process used is capable of repeating a consistent form, i.e. not a random process, in that the relative movement between the device and the power beam can be controlled and repeated.

In one embodiment a plurality of power beams can be used.

As the process uses the parent material to form the textured surface there is no corrosion potential and no potential for contamination.

In one embodiment the component includes at least two portions, each having textures with differing features/densities for the different portions. In one embodiment for bone growth the surface finish texture on an implant can be modified to match the varying density of bone through its cross section.

In one embodiment, the device includes an external surface with a relatively complex, spiked, form in order to aid fixation of the device to bone during a surgical implant technique to reduce or eliminate micro-motion of the implant during use and service, which movement would otherwise hinder bone growth onto the implant.

In one embodiment, the device includes a rail to allow the implant to be press fitted in position and said rail is provided with a surface finish utilising the invention. This embodiment allows the disadvantages of bead sintered femoral knee implants which have restricted use for press fit applications as the required bead rail, necessary for retention of beads, prevents the beaded surface from being pressed up against bone during implantation, to be overcome as the rail is textured to allow press fit application of the bone growth surfaces.

In one embodiment, the device includes an external implant rasping surface with a series of cutting ridges/loops which would as well as acting as a bone growth surface also create chippings of bone during surgical insertion that would act as bone impaction material to improve fixation for cementless applications.

Typically as a result of this power beam process, it is possible to texture a surface pre-coated with a bioactive coating such as calcium phosphate or hydroxypatite. This improves the bonding between a growth enhancing coating and the base component, possibly resulting in a composite texture that promotes bone growth and hence secure the implant more readily than with the base component material alone.

In one embodiment the portion of the device incorporates a roughened surface with indents and asperities <50 micron. Alternatively the surface finish of the portion can be controlled to produce smooth continuous surfaces in regions of high tensile stresses, thus improving the fatigue resistance of the portion and the device as a whole.

In one embodiment the modification is achieved in the same material from which the device is formed.

In one embodiment of the invention there is provided a medical device, said device having a body portion and wherein a portion of the external surface of the body portion is modified by exposing the same to a power beam in a controlled manner, said surface portion provided in use of the device in contact with a body part of a patient to aid the fixing and/or subsequent bonding of the device as an integral part of the patient's bone structure.

In one embodiment the device is an implant and the said surface portion includes a first formation which is provided to aid the ongrowth of bone onto the implant when fitted in position, and at least a second formation to aid the retention of the device in position.

In a further embodiment of the invention when the medical device is a tool, the device includes a first portion which is used as working portion and a second portion used as a handle and wherein at least one of said portions has a surface modified by exposure of the same to a power beam in a controlled manner. Preferably both of said portions have modified surface textures by exposure to a power beam in a controlled manner and typically the modification and surface shapes formed on the working portion differ to those formed on the handle portion such that the surfaces can be provided with the appropriate formation to aid use of the device, without the need for additional material to be added to the device body.

Specific embodiments of the invention are now described with reference to the accompanying drawings; wherein

FIGS. 1a-c illustrate a modified surface in accordance with a first embodiment of the invention; and

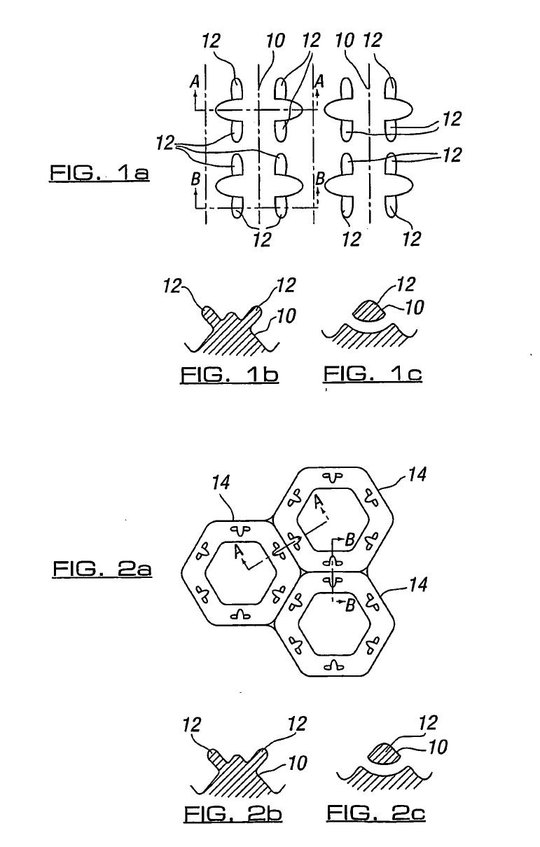

FIGS. 2a-c illustrate a variation on the modified surface of FIGS. 1a-c in accordance with another embodiment of the invention;

FIGS. 3a-b illustrate bone growth surfaces in an open porous network, in one embodiment;

FIGS. 4a-b illustrate a modified surface in accordance with another embodiment of the invention;

FIGS. 5a-b illustrate a modified surface with no mechanical interlocking features;

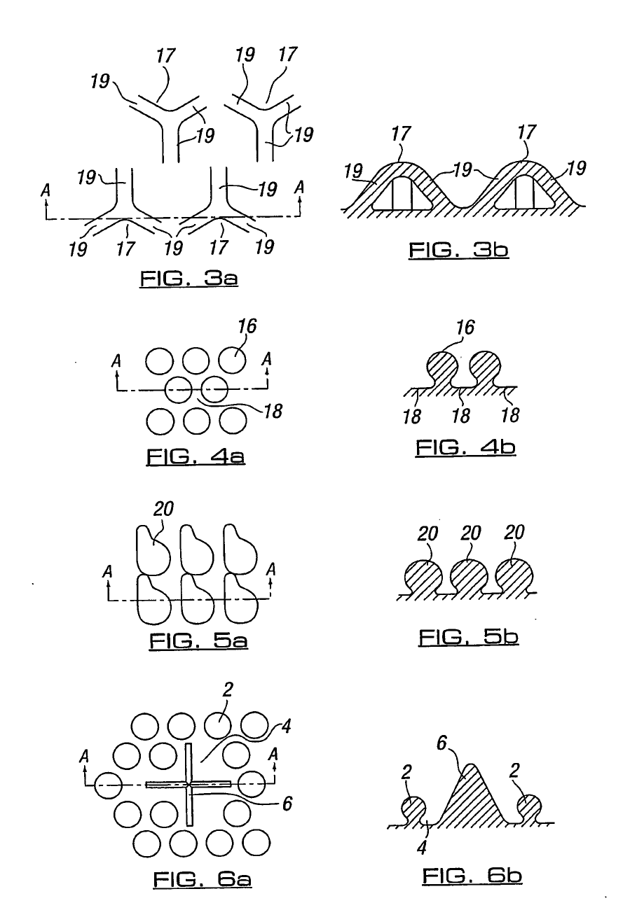

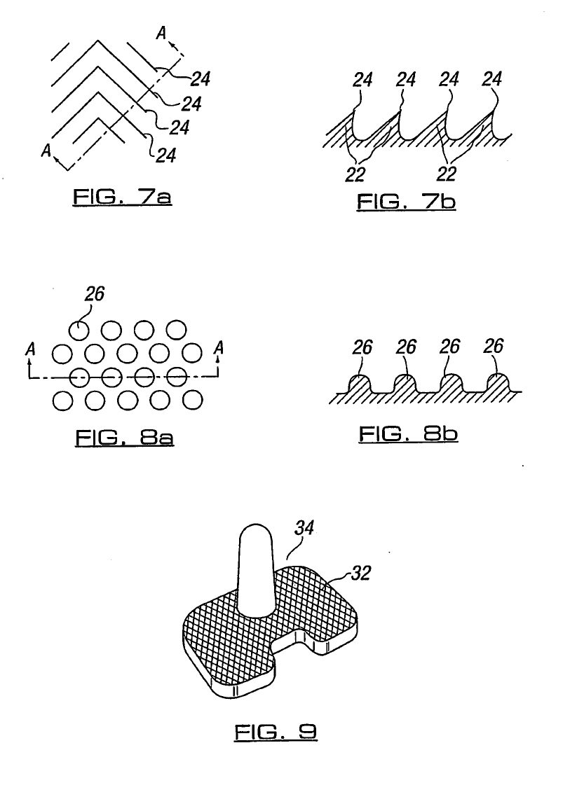

FIGS. 6a and b illustrate a modified surface with mechanical fixation features;

FIGS. 7a and b illustrate a modified surface for use with a medical tool in one embodiment;

FIGS. 8a and b illustrate a modified surface configuration for use as a handle of a medical tool;

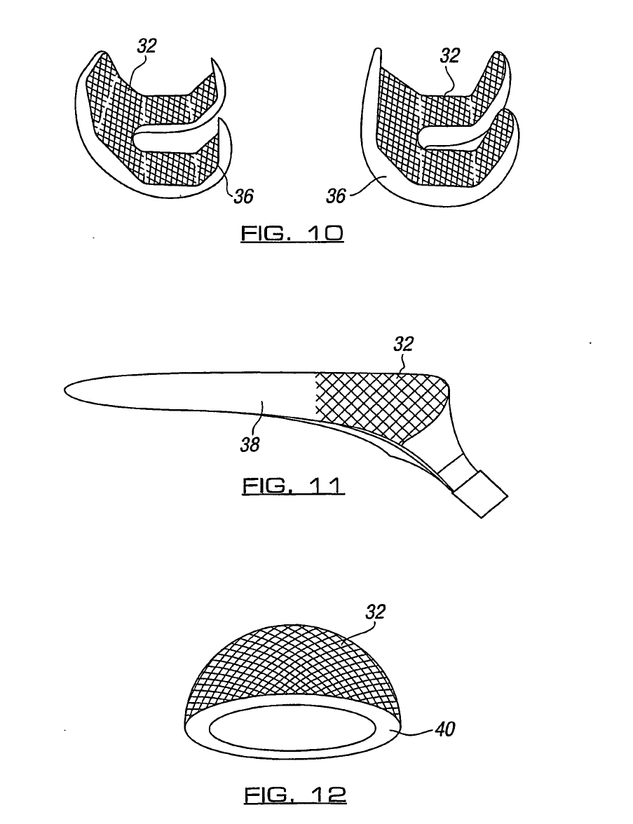

FIGS. 9-12 illustrate various designs of a medical device in the form of an implant including a portion with a modified surface achieved in accordance with the invention;

FIGS. 13a and b illustrate a further example of a modified portion to a form bone growth surfaces with an open porous network; and

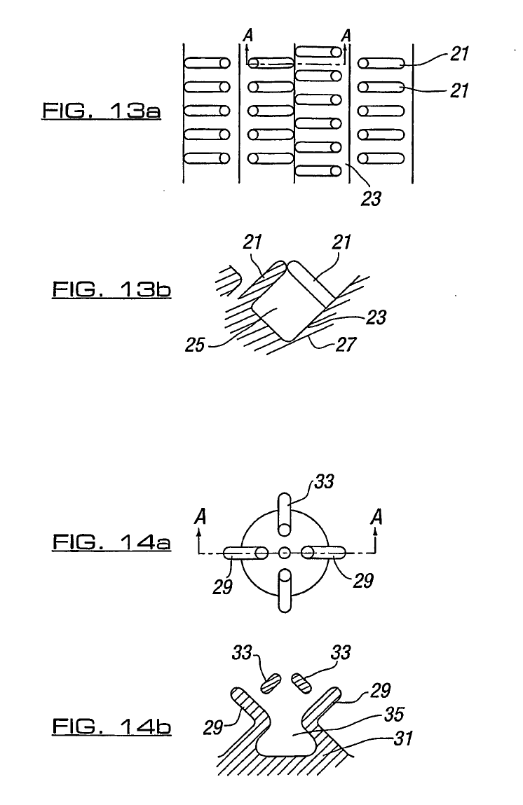

FIGS. 14a-b illustrate a yet further example of a modified portion of a surface of a device which has been modified in accordance with the invention to form a bone growth surface with an open porous network.

All of the figures herein described illustrate modified surface finishes which can be achieved on medical devices in accordance with the invention by exposing the surfaces to be modified to a power beam in a controlled manner.

FIG. 1a-c illustrate a modified surface. In this case, improved bone oseointegration is an aim and there are provided a series of raised portions 10 which are provided in a relatively complex arrangement and with angled protrusions 12. This particular pattern has been developed to encourage bone ongrowth and the labyrinth growth pattern through and between the protrusions is also found to improve good bone vascularity.

FIGS. 2a-c illustrate a variation on the configuration of FIGS. 1a-c where the pattern is formed with a plurality of cells 14.

FIG. 3a and b illustrate another example of a surface finish with a bone growth surface in an open porous network form in which there are provided a series of portions 17 with arms or limbs 19, in a Y-shaped formation.

In FIGS. 4a and b there is shown a further variation in which a series of nodules 16 are provided and spaced by a recessed array 18, without a forming location.

In FIGS. 13a and b there is shown a further embodiment of an open porous network bone growth surface which has been formed in accordance with the invention. In this case the surface comprises a series of limbs 21 which protrude from the surface 23 thereby forming a series of cavities and pores 25 into which the bone can grow and thereby integrate the implant device 27.

FIG. 14a and b illustrate a further embodiment of the invention in which there is provided another form of surface which includes protrusions 29 depending upwardly from the surface 31 of the device. Longer protrusions 33 are provided which in combination again form the recesses 35 into which the bone can grow.

In FIGS. 5a and b there is illustrated a textured surface finish in which there are provided a series of protrusions 20 shaped so as to provide an encouragement to bone growth but, in this case without the need for mechanical interlocking.

In FIGS. 6a-b there is shown a plan view of part of a surface finish of a design which can be used on an implant. The surface includes a plurality of nodules 2 in a spaced pattern and spaced by a recessed array 4. There is also provided a raised, cross shaped feature 6. In this case the modified surface is provided as a portion of the external surface of a medical device in the form of an implant for a hip or knee joint. In this case the nodule and recess array 2, 4 is provided in a form to encourage the growth of the patient's bone around the nodules once the insert is in position thereby improving the ability for the implant to be accepted by the patient and be integrated into their bone structure as quickly as possible.

The shaped feature 6 is provided as a fixation feature to allow the implant to be securely attached to and retained in position in the patient's hip or knee at the time of surgery such that in this case the implant has fixation and ongrowth surface portions.

FIGS. 7c and b illustrate a function which can be provided to allow a tool, in this case a rasp to be formed in accordance with the invention in which a series of ridges 22 with rasping edges 24 are formed on the rasping surface. FIGS. 8a and 8b illustrate a handle formation which can also be provided on the rasp of FIGS. 7a and b at a different portion, or on other tools, to allow the handle to be efficiently grasped. In this case a series of circular portions 26 are raised in a configuration to provide improved gripping. In a preferred embodiment the tool includes both types of formations as shown at different locations.

It should therefore be appreciated that a wide range of surface modification patterns can be achieved in the same material from which the medical device is formed and that different configurations can be provided on different portions of the same medical device without the addition of other materials.

FIGS. 9-12 illustrate a series of medical devices each of which include a portion 32 of an outer surface thereof which has been modified by exposing that surface to the power beam in a controlled manner. In FIG. 9 there is shown a fabric tibia implant 34 in which the plate surface 34 has been provided with a modified surface in accordance with the invention.

In FIG. 10 there are shown two femoral knee implants 36, with portions 32 modified in accordance with the invention.

In FIG. 11 there is shown a total femoral hip implant 38 in which an intermediate surface portion has been modified.

In FIG. 12 there is shown an acetabular cup hip implant 40 in which the upper surface 32 has been modified.

In each case the modification has been achieved by exposing the surfaces to one or more power beams with relative movement in a controlled path. This allows the surface to be modified and avoids the need for other materials to be added or joined to the base, thereby avoiding the problems which this eventually causes.

All of this can be achieved by the exposure of the selected portions of the device to a power beam, typically a high power beam. The control of the power beam and relative movement of the same and the device allow the formation of the protrusions, nodules and recessed arrays to provide exact and relatively complex surface finishes to allow the same to be tailored to suit the specific purpose of the particular medical device. Indeed the device can be provided with a plurality of surface portions which are modified using power beam exposure and each of said portions can be provided with a finish which can be tailored to the needs for that particular portion, such that the surface finish on one portion may differ to other portions on the same device. Indeed it is also possible that coatings of particular materials can be applied to the device which will encourage the growth of bone onto the device if required and the surfaces can still be modified as herein described.

The present invention also allows the manufacture to be achieved using a low temperature process thus being able to produce a porous surface without altering the as-cast microstructure and hence maintaining the desirable wear characteristics.

1. A medical device, wherein said device includes at least a portion of its external surface formed into a predetermined configuration by the exposure of said portion to a power beam and relative movement between the power beam and said device is controlled to form the said external surface into the predetermined configuration.

2. A device according to claim 1 wherein the power beam is an electron beam or a laser beam.

3. A device according to claim 1 wherein the said external surface includes any or any combination of protrusions and/or pores and/or indents.

4. A device according to claim 1 wherein the device is initially formed and the surface portion of the same is subsequently modified by exposure to the power beam.

5. A device according to claim 1 wherein the portion of the device which is modified is formed of the same material as the remainder of the medical device.

6. A device according to claim 1 wherein the material of the portion which is exposed to the power beam is melted and forms a welded interface between the surface and the remainder of the device.

7. A device according to claim 1 wherein the device is an implant.

8. A device according to claim 7 wherein the surface of the portion is formed so as to encourage the growth and anchorage of bone to the implant and/or to improve the vascular properties of the same.

9. A device according to claim 1 wherein the device is a surgical tool and the surface portion is formed so as to perform a working action.

10. A device according to claim 9 wherein the working action is cutting or rasping.

11. A device according to claim 9 wherein the portion exposed to the power beam is the handle of the device.

12. A device according to claim 1 wherein the device is an implant and the at least one portion is formed to have any or any combination of the following features of pores and/or re-entrant features and/or undercut features to provide macro-interlocking of the bone and the implant device.

13. A device according to claim 12 wherein the pores are of the range of ˜1 mm in size.

14. A device according to claim 1 wherein the device is an implant and the at least one portion is formed with micro pores suitable for the incorporation of bone trabecula.

15. A device according to claim 14 wherein the pores are of a size in the range of 100-200 microns.

16. A device according to claim 1 wherein the device is an implant and the at least one portion includes pores suitable for the attachment of bone building cells (osteoclasts & osteoblasts).

17. A device according to claim 16 wherein the pores are of a size in the range of 10-20 microns.

18. A medical device which has at least a portion of its external surface modified after the device has been initially formed, said modification achieved by the selective exposure of said portion to a power beam, and controlled relative movement between the beam and said portion.

19. A device according to claim 1 wherein the device includes a portion of the external surface with a complex, spiked configuration in order to aid fixation of the device to bone during a surgical implant technique.

20. A device according to claim 1 wherein the device includes a rail to allow the implant to be press fitted in position and said rail is provided with a portion which is exposed to the power beam.

21. A device according to claim 1 wherein the device includes an external implant rasping surface with a series of cutting ridges/loops.

22. A device according to claim 1 wherein the device includes a surface coated with a bone growth enhancing material which is then exposed to the power beam to modify the same.

23. A device according to claim 1 wherein the device has a body and wherein a portion of the external surface of the body portion is modified by exposing the same to a power beam in a controlled manner, said surface portion provided in use of the device in contact with a body part of a patient to aid the fixing and/or subsequent bonding of the device as an integral part of the patient's bone structure.

24. A device according to claim 23 wherein the device is an implant and the said surface portion includes a first formation which is provided to aid the ongrowth of bone onto the implant when fitted in position, and at least a second formation to aid the retention of the device in position.

25. A device according to claim 1 wherein the device is a tool, the device includes a first part which is used as a working portion and a second part used as a handle and wherein at least one of said portions has a surface modified by exposure of the same to a power beam in a controlled manner.

26. A device according to claim 25 wherein both of said portions have modified surfaces as a result of exposure to a power beam in a controlled manner.

27. A device according to claim 26 wherein the modification and surface shapes formed on the working portion differ to those formed on the handle portion.

28. A method of forming a medical device, said method comprising the steps of forming the device from a metal or metal alloy into an initial form and wherein at least a portion of the surface is then exposed to a power beam and relative movement between the device and power beam causes modification of the material of the portion to create a surface finish of the desired form.

29. A method according to claim 28 wherein the material exposed to the power beam is melted.

30. A method according to claim 29 wherein the remainder of the device which is not exposed to the power beam remains at temperatures that do not negatively affect the mechanical properties of the device.

31. A method according to claim 29 wherein following the melting of the material rapid solidification results in a textured finish of the portion surface with a fine grained and homogenous microstructure.

32. A method according to claim 28 wherein a plurality of power beams are used.

33. A method according to claim 28 wherein at least two portions of the surface of the device are modified.

34. A method according to claim 28 wherein the surface finish of the said portion is modified to match the varying density of bone through its cross section.
